(19) United States
(12) Patent Application Publication
(10) Pub. No.: US 2024/0108892 A1
(43) Pub. Date: Apr. 4, 2024

(54) Changing the Orientation of Tumor Treating Fields (TTFields) by Adjusting the Amplitudes of Two or More Electric Fields that Are All In-Phase with Each Other

(71) Applicant: Novocure GmbH, Root D4 (CH)

(72) Inventors: Michael MODILEVSKY, Haifa (IL); Yevgeniy TELEPINSKY, Haifa (IL); Michael KRINITSKY, Haifa (IL)

(73) Assignee: Novocure GmbH, Root D4 (CH)

(21) Appl. No.: 18/374,082

(22) Filed: Sep. 28, 2023

Related U.S. Application Data
(60) Provisional application No. 63/411,810, filed on Sep. 30, 2022.

(51) Int. Cl. A61N 1/36; A61N 1/04

(52) U.S. Cl. CPC A61N 1/36002; A61N 1/04

(57) ABSTRACT

Tumor Treating Fields (TTFields) therapy is a proven approach for treating tumors using alternating electric fields. When treating tumors in certain anatomic locations (e.g., at certain locations within a subject's head) it may be beneficial to induce an electric field through the subject's body with an overall directionality that varies between more than two directions. The embodiments described herein can induce electric fields in many different directions by simultaneously applying AC signals to different pairs of transducer arrays and adjusting the amplitudes of those AC signals.

### CROSS REFERENCE TO RELATED APPLICATIONS

This application claims the benefit of U.S. Provisional Application 63/411,810, filed Sep. 30, 2022, which is incorporated herein by reference in its entirety.

### BACKGROUND

Tumor Treating Fields (TTFields) therapy is a proven approach for treating tumors using alternating electric fields at frequencies between 50 kHz and 1 MHz (e.g., 150-200 kHz). In the prior art Optune® system, TTFields are delivered to patients via four transducer arrays that are placed on the patient's skin near the tumor. The transducer arrays are arranged in two pairs, with one pair of transducer arrays positioned to the left and right of the tumor, and the other pair of transducer arrays positioned anterior and posterior to the tumor. Each transducer array is connected via a multi-wire cable to an AC signal generator. The AC signal generator (a) sends an AC current through the left/right (L/R) pair of transducer arrays for 1 second, which induces an electric field with a first direction through the tumor; then (b) sends an AC current through the anterior/posterior (A/P) pair of arrays for 1 second, which induces an electric field with a second direction through the tumor; then repeats steps (a) and (b) for the duration of the treatment. Each transducer array includes a plurality (e.g., between 9 and 30) of electrode elements.

Alternating electric fields can also be used to treat medical conditions other than tumors. For example, as described in U.S. Pat. No. 10,967,167 (which is incorporated herein by reference in its entirety), alternating electric fields can be used to increase the permeability of the blood brain barrier (BBB) so that, e.g., chemotherapy drugs can reach the brain.

FIG. 1 depicts the AC signal generator 12 of the prior art Optune® system, and FIG. 2 is a schematic representation of the top view of the Optune® system's A/P and L/R transducer arrays 10 positioned on a person's head. Notably, in the prior art Optune® system, only one of the outputs Q1, Q2 is active at any given instant. When output Q1 is active, the AC signal generator 12 sends an AC current through the L/R pair of transducer arrays, and an electric field with field lines 21 is induced through the tumor (as seen in the left half of FIG. 2). And when output Q2 is active, the AC signal generator 12 sends an AC current through the A/P pair of transducer arrays, and an electric field with field lines 22 is induced through the tumor (as shown in the right half of FIG. 2).

FIG. 3 is a schematic representation of the overall directionality of the electric fields 21, 22 shown in FIG. 2. More specifically, when output Q1 is active (and the AC signal generator 12 is sending an AC current through the L/R pair of transducer arrays 10L/10R), an electric field with a first overall direction 31 is induced. And when output Q2 is active (and the AC signal generator 12 is sending an AC current through the A/P pair of arrays 10A/10P), an electric field with a second overall direction 32 is induced.

Notably, when the prior art Optune® AC signal generator 12 is used together with the prior art transducer arrays 10, only the two overall directions depicted in FIG. 3 are obtainable, and it is not possible to induce an electric field with different overall directionality (e.g., a diagonal orientation) through the subject's head.

### SUMMARY OF THE INVENTION

One aspect of the invention is directed to a first method of applying an alternating electric field to a target region in a subject's body using a first set of one or more first electrode elements positioned on a first side of the target region, a second set of one or more second electrode elements positioned on a second side of the target region that is opposite to the first side, a third set of one or more third electrode elements positioned on a third side of the target region, and a fourth set of one or more fourth electrode elements positioned on a fourth side of the target region that is opposite to the third side. The first method comprises inducing a first component of an electric field by applying a first AC signal between the first set of one or more first electrode elements and the second set of one or more second electrode elements and inducing a second component of an electric field by applying a second AC signal between the third set of one or more third electrode elements and the fourth set of one or more fourth electrode elements. The first and second AC signals are applied simultaneously, and the first and second AC signals are in phase with each other. The first method also comprises varying an amplitude of at least one of the first AC signal and the second AC signal over time such that an orientation of an alternating electric field formed by superposition of the first component and the second component varies over time.

Some instances of the first method further comprise positioning the first set of one or more first electrode elements on the first side of the target region; positioning the second set of one or more second electrode elements on the second side of the target region; positioning the third set of one or more third electrode elements on the third side of the target region; and positioning the fourth set of one or more fourth electrode elements on the fourth side of the target region. Optionally, all of the electrode elements are capacitively coupled to the subject's body.

Some instances of the first method further comprise positioning the first set of one or more first electrode elements on the first side of the target region; positioning the second set of one or more second electrode elements on the second side of the target region; positioning the third set of one or more third electrode elements on the third side of the target region; and positioning the fourth set of one or more fourth electrode elements on the fourth side of the target region. All of the electrode elements are conductively coupled to the subject's body.

In some instances of the first method, the varying comprises varying the amplitudes of both the first AC signal and the second AC signal over time so that that the orientation of the alternating electric field rotates. In some instances of the first method, the varying comprises varying the amplitude of at least one of the first AC signal and the second AC signal over time so that the orientation of the alternating electric field oscillates back and forth.

In some instances of the first method, the varying comprises repeating the following steps in an alternating sequence at least 1000 times: setting the amplitudes of the first AC signal and the second AC signal so that the amplitude of the first AC signal is greater than the amplitude of the second AC signal; and setting the amplitudes of the first AC signal and the second AC signal so the amplitude of the second AC signal is greater than the amplitude of the first AC signal.

In some instances of the first method, the varying comprises repeating the following steps in an alternating sequence at least 1000 times: increasing the amplitude of the first AC signal from a minimum value to a maximum value; and decreasing the amplitude of the first AC signal from a maximum value to a minimum value.

Some instances of the first method further comprise inducing a third component of an electric field by applying a third AC signal between a fifth set of one or more fifth electrode elements positioned on a fifth side of the target region and a sixth set of one or more sixth electrode elements positioned on a sixth side of the target region that is opposite to the fifth side. In these instances, the first, second, and third AC signals are all applied simultaneously, and the first, second, and third AC signals are all in phase with each other. An amplitude of the first, second, and third AC signals are varied over time such that an orientation of a vector representing a superposition of the first component, the second component, and the third component varies over time in more than two dimensions.

Another aspect of the invention is direct to a first apparatus for applying an alternating electric field to a target region in a subject's body using a first set of one or more first electrode elements positioned on a first side of the target region, a second set of one or more second electrode elements positioned on a second side of the target region that is opposite to the first side, a third set of one or more third electrode elements positioned on a third side of the target region, and a fourth set of one or more fourth electrode elements positioned on a fourth side of the target region that is opposite to the third side. The first apparatus comprises an AC signal generator and a controller. The AC signal generator is configured to apply a first AC signal between the first set of one or more first electrode elements and the second set of one or more second electrode elements and to simultaneously apply a second AC signal between the third set of one or more third electrode elements and the fourth set of one or more fourth electrode elements, wherein the first and second AC signals are in phase with each other, and wherein the AC signal generator is configured to adjust an amplitude of the first AC signal and to adjust an amplitude of the second AC signal based on control signals that arrive at at least one control input. The controller is configured to send a sequence of control signals to the at least one control input, wherein the control signals cause the AC signal generator to vary the amplitude of at least one of the first AC signal and the second AC signal over time so that an orientation of an alternating electric field that is induced in the target region due to (a) application of the first AC signal between the first set of one or more first electrode elements and the second set of one or more second electrode elements and (b) application of the second AC signal between the third set of one or more third electrode elements and the fourth set of one or more fourth electrode elements varies over time.

Some embodiments of the first apparatus further comprise the first set of one or more first electrode elements; the second set of one or more second electrode elements; the third set of one or more third electrode elements; and the fourth set of one or more fourth electrode elements. Optionally, in these embodiments, all of the electrode elements are capacitively coupled to the subject's body.

Some embodiments of the first apparatus further comprise the first set of one or more first electrode elements; the second set of one or more second electrode elements; the third set of one or more third electrode elements; and the fourth set of one or more fourth electrode elements. All of the electrode elements are conductively coupled to the subject's body.

In some embodiments of the first apparatus, the control signals cause the AC signal generator to vary the amplitude of both the first AC signal and the second AC signal over time so that that the orientation of the alternating electric field rotates. In some embodiments of the first apparatus, the control signals cause the AC signal generator to vary the amplitude at least one of the first AC signal and the second AC signal over time so that the orientation of the alternating electric field oscillates back and forth.

In some embodiments of the first apparatus, the control signals cause the AC signal generator to repeat the following steps in an alternating sequence at least 1000 times: setting the amplitudes of the first AC signal and the second AC signal so that the amplitude of the first AC signal is greater than the amplitude of the second AC signal; and setting the amplitudes of the first AC signal and the second AC signal so the amplitude of the second AC signal is greater than the amplitude of the first AC signal.

In some embodiments of the first apparatus, the control signals cause the AC signal generator to repeat the following steps in an alternating sequence at least 1000 times: increasing the amplitude of the first AC signal from a minimum value to a maximum value; and decreasing the amplitude of the first AC signal from a maximum value to a minimum value.

In some embodiments of the first apparatus, the AC signal generator is further configured to apply a third AC signal between a fifth set of one or more fifth electrode elements positioned on a fifth side of the target region and a sixth set of one or more sixth electrode elements positioned on a sixth side of the target region that is opposite to the fifth side, wherein the first, second, and third AC signals are all in phase with each other, and the AC signal generator is further configured to adjust an amplitude of the third AC signal based on additional control signals that arrive at the at least one control input. In these embodiments, the controller is further configured to send the additional control signals to the at least one control input, wherein the control signals and the additional control signals collectively cause the AC signal generator to vary the amplitude of the first, second, and third AC signals over time so that an orientation of an alternating electric field that is induced in the target region due to application of the first, second, and third AC signals varies over time in more than two dimensions.

### BRIEF DESCRIPTION OF THE DRAWINGS

FIG. 1 depicts the AC signal generator of the prior art Optune® system.

FIG. 2 is a schematic representation of the top view of the Optune® system's transducer arrays positioned on a person's head.

FIG. 3 is a schematic representation of the overall directionality of the electric fields shown in FIG. 2.

FIG. 4 is a block diagram of a system that can induce electric fields in more than two directions by simultaneously applying AC signals to two or more pairs of transducer arrays.

FIG. 5 depicts four examples of how the overall directionality of the electric field can vary when using the FIG. 4 embodiment.

FIG. 6 depicts additional details that correspond to the top left panel of FIG. 5.

FIG. 7 depicts additional details that correspond to the top right panel of FIG. 5.

FIG. 8 depicts additional details that correspond to the lower left panel of FIG. 5.

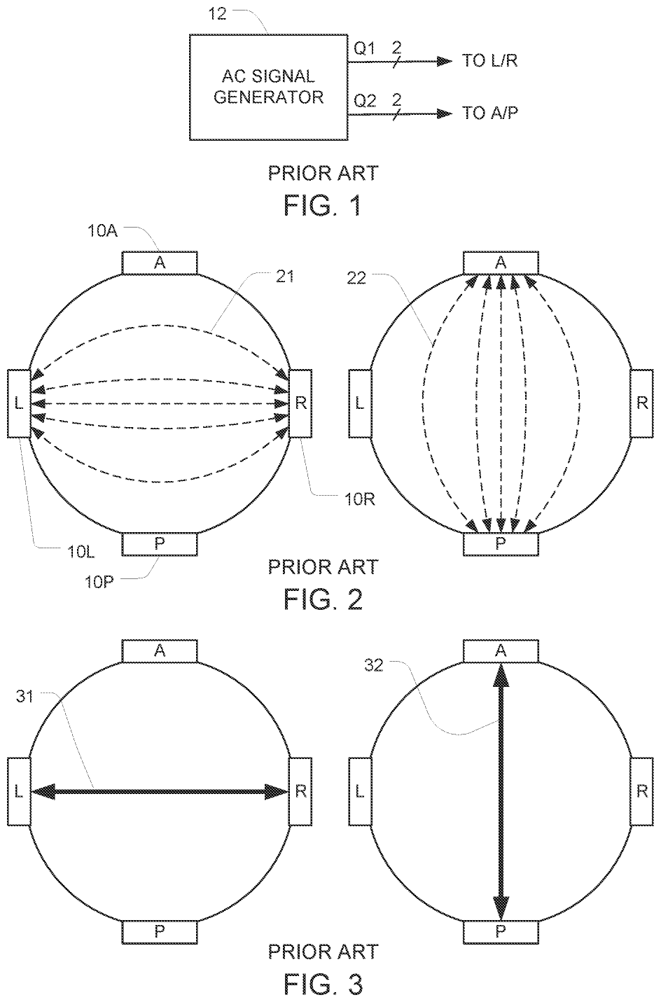

Various embodiments are described in detail below with reference to the accompanying drawings, wherein like reference numerals represent like elements.

### DESCRIPTION OF THE PREFERRED EMBODIMENTS

When treating tumors in certain anatomic locations (e.g., at certain locations within a subject's head) it may be beneficial to induce an electric field through the subject's body with an overall directionality that is different from directions 31 and 32 depicted in FIG. 3. The embodiments described herein can induce electric fields in those different directions by simultaneously applying first and second AC signals that are in-phase with each other to the L/R and A/P pairs of transducer arrays, respectively, and adjusting the amplitudes of those two AC signals.

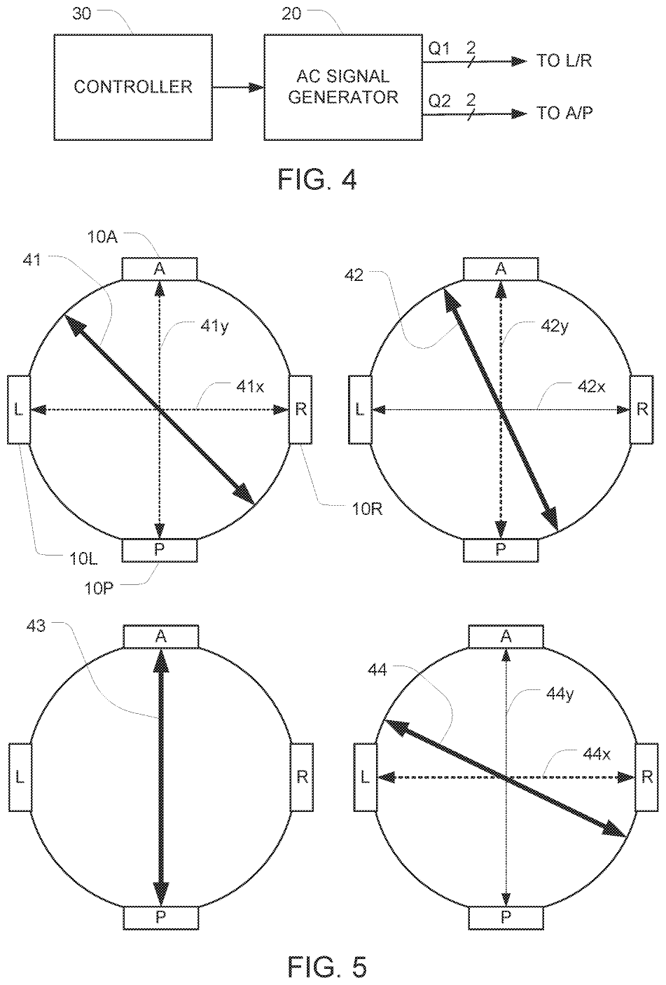

FIG. 4 is a block diagram of a system that can induce electric fields in more than two directions by simultaneously applying AC signals to two or more pairs of transducer arrays. The system includes an AC signal generator 20 that has two outputs Q1 and Q2. One of those outputs Q1 drives the left and right transducer arrays 10L/10R, and the other output Q2 drives the anterior and posterior transducer arrays 10A/10P. The transducer arrays 10 in this FIG. 4 embodiment are similar to the prior art transducer arrays, and are positioned similarly on the subject's body. Notably, unlike the prior art Optune® system in which only one output of the AC signal generator 12 is active at any given instance, both outputs Q1, Q2 of the AC signal generator 20 in the FIG. 4 embodiment can be active simultaneously. The amplitudes of these outputs Q1, Q2 are individually adjustable, and the AC signal generator 20 is configured to individually adjust these outputs Q1, Q2 based on commands received from a controller 30.

We will now examine what happens in a number of situations when the amplitude of the outputs Q1, Q2 of the AC signal generator 20 are set to different levels.

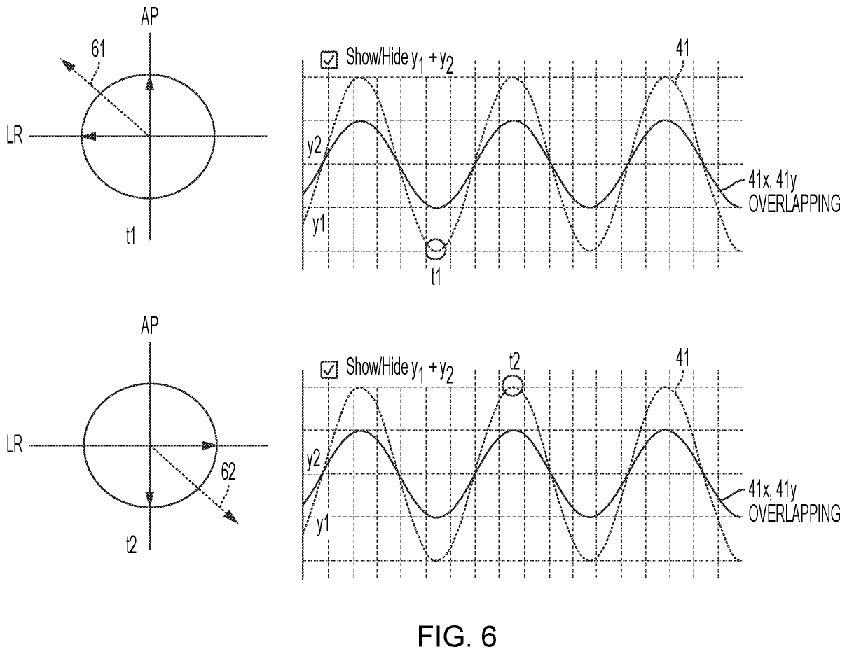

The top left quadrant of FIG. 5 depicts the overall directionality of the electric field that is induced through the subject's body when both outputs Q1 and Q2 of the AC signal generator 20 are active simultaneously at the same amplitude. In this situation, a first AC signal that is applied to the left and right transducer arrays 10L, 10R will induce a first component of an electric field with an overall directionality 41x, and a second AC signal that is applied to the anterior and posterior transducer arrays 10A, 10P will induce a second component of an electric field with an overall directionality 41y. The first and second AC signals are applied simultaneously via Q1 and Q2 and are in phase with each other. In this situation, the orientation of an alternating electric field that will be induced in the subject's body is formed by superposition of the first component 41x and the second component 41y, and the resulting superposition will have the overall directionality 41 for the reasons explained in more detail below in connection with FIG. 6.

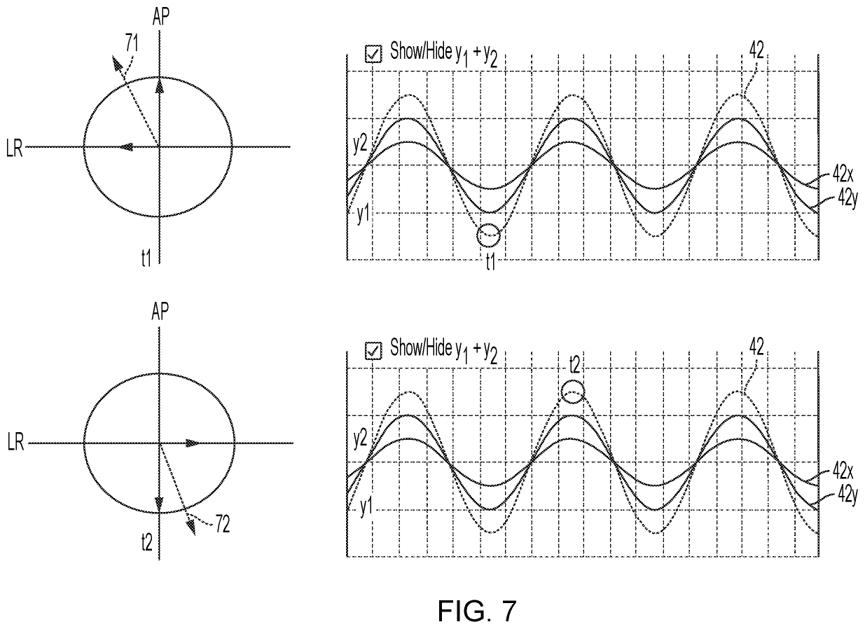

The top right quadrant of FIG. 5 depicts the overall directionality of the electric field that is induced through the subject's body when both outputs Q1 and Q2 of the AC signal generator 20 are active simultaneously, but the amplitude of Q1 is half the amplitude of Q2. In this situation, a first AC signal that is applied to the left and right transducer arrays 10L, 10R will induce a first (smaller) component of an electric field with an overall directionality 42x, and a second AC signal that is applied to the anterior and posterior transducer arrays 10A, 10P will induce a second (larger) component of an electric field with an overall directionality 42y. The first and second AC signals are applied simultaneously via Q1 and Q2 and are in phase with each other. In this situation, the orientation of an alternating electric field that will be induced in the subject's body is formed by superposition of the first component 42x and the second component 42y, and the resulting superposition will have the overall directionality 42 for the reasons explained in more detail below in connection with FIG. 7.

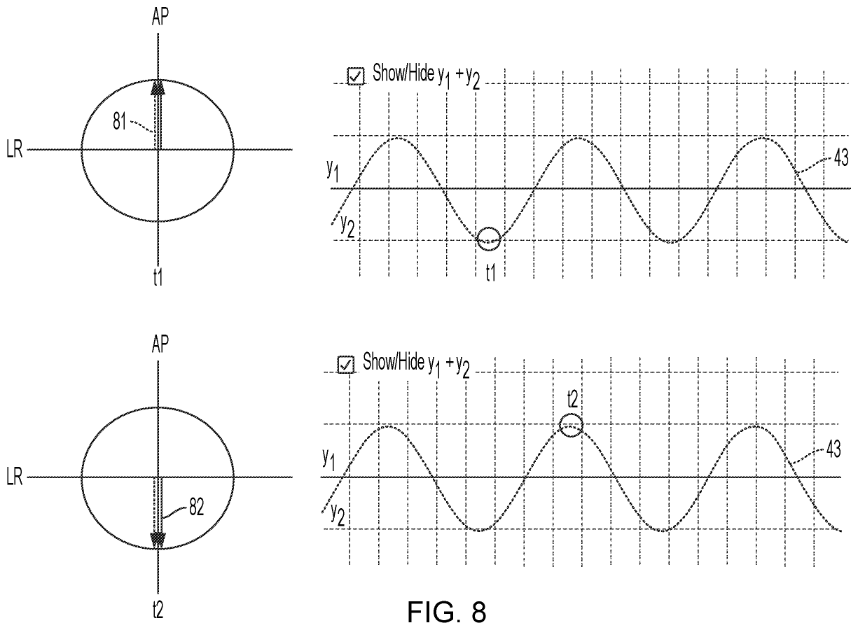

The lower left quadrant of FIG. 5 depicts the overall directionality of the electric field that is induced through the subject's body when only output Q2 of the AC signal generator 20 is active, and the amplitude of output Q1 is set to zero. In this situation, no signal is applied to the left and right transducer arrays 10L, 10R, and an AC signal is applied only to the anterior and posterior transducer arrays 10A, 10P. This will induce an electric field with an overall directionality 43 for the reasons explained in more detail below in connection with FIG. 8. (Note that this yields an end result that is similar to the situation described above in connection with the right half of FIG. 3.)

The lower right quadrant of FIG. 5 depicts the overall directionality of the electric field that is induced through the subject's body when both outputs Q1 and Q2 of the AC signal generator 20 are active simultaneously, but the amplitude of Q1 is twice the amplitude of Q2. In this situation, a first AC signal that is applied to the left and right transducer arrays 10L, 10R will induce a first (larger) component of an electric field with an overall directionality 44x, and a second AC signal that is applied to the anterior and posterior transducer arrays 10A, 10P will induce a second (smaller) component of an electric field with an overall directionality 44y. The first and second AC signals are applied simultaneously via Q1 and Q2 and are in phase with each other. Once again, the orientation of an alternating electric field that will be induced in the subject's body is formed by superposition of the first component 44x and the second component 44y, and the resulting superposition will have the overall directionality 44.

Note that while the top left, top right, and lower right quadrants of FIG. 5 each depicts the situation in which the two components (e.g., 41x and 41y in the top left quadrant) that ultimately add by superposition into a resulting sum with an overall directionality (e.g., 41 in the top left quadrant) are orthogonal. But orthogonality is not a prerequisite for the two components to add by superposition. Therefore, even if the L/R pair of electrodes is offset from orthogonal with respect to the A/P pair of electrodes, the components that are attributable to each of those pairs of electrodes will still add by superposition into a sum that has a single overall directionality. The math for computing the resulting field direction will, however, be somewhat more difficult in order to account for any offset from orthogonality.

FIG. 6 depicts additional details that correspond to the top left panel of FIG. 5. Because both outputs Q1 and Q2 are in phase and have the same amplitude, the traces for those two outputs 41x, 41y overlap on FIG. 6. The superposition of those two traces 41x, 41y results in the trace 41. Notably, at time t1, there is a negative component in the x direction and a negative component in the y direction, which results in an instantaneous superposition 61. Similarly, at time t2, there is a positive component in the x direction and a positive component in the y direction, which results in an instantaneous superposition 62.

FIG. 7 depicts additional details that correspond to the top right panel of FIG. 5, when both outputs are active simultaneously, but the amplitude of Q1 (trace 42x) is half the amplitude of Q2 (trace 42y). The superposition of those two traces 42x, 42y results in the trace 42. Notably, at time t1, there is a negative component in the x direction and a larger negative component in the y direction, which results in an instantaneous superposition 71. Similarly, at time t2, there is a positive component in the x direction and a larger positive component in the y direction, which results in an instantaneous superposition 72.

FIG. 8 depicts additional details that correspond to the lower left panel of FIG. 5. In this situation, no signal (i.e., zero amplitude) is applied to the left and right transducer arrays 10L, 10R, so there is no x component. There is only a y component, so the superposition (trace 43) is the same as the y component. Notably, at time t1, there is a negative component in the y direction, which results in an instantaneous superposition 81. Similarly, at time t2, there is a positive component in the y direction, which results in an instantaneous superposition 82.

The ability to steer the overall directionality of the electric field to any desired direction can provide additional flexibility in terms of deciding where to position the transducer arrays on a given subjects body's during a treatment-planning phase that precedes the application of the alternating electric fields. For example, if a large part of a particular subject's forehead is afflicted by a skin condition that makes it painful to position a transducer array there, it could be impossible to generate electric fields in two different directions that are roughly orthogonal using prior art treatment-planning techniques. In contrast, by using the ability to steer the overall directionality of the electric field provided by the embodiments described herein, all four transducer arrays could be evenly distributed around the sides and back of the subject's head, and it will still be possible to generate electric fields in two different directions that are roughly orthogonal (which can provide improved results).

Returning to FIGS. 4 and 5, the apparatus 10, 20 applies an alternating electric field to a target region in a subject's body using a first set of one or more first electrode elements 10L positioned on a first side of the target region, a second set of one or more second electrode elements 10R positioned on a second side of the target region that is opposite to the first side, a third set of one or more third electrode elements 10A positioned on a third side of the target region, and a fourth set of one or more fourth electrode elements 10P positioned on a fourth side of the target region that is opposite to the third side. The apparatus includes an AC signal generator 20 and a controller 30.

The AC signal generator 20 is configured to apply a first AC signal between the first set of one or more first electrode elements 10L and the second set of one or more second electrode elements 10R and to simultaneously apply a second AC signal between the third set of one or more third electrode elements 10A and the fourth set of one or more fourth electrode elements 10P. The first and second AC signals are in phase with each other, and the AC signal generator 20 is configured to adjust an amplitude of the first AC signal and to adjust an amplitude of the second AC signal based on control signals that arrive at at least one control input.

The controller 30 is configured to send a sequence of control signals to the at least one control input, so that the control signals cause the AC signal generator 20 to vary the amplitude of at least one of the first AC signal and the second AC signal over time so that an orientation of an alternating electric field that is induced in the target region due to (a) application of the first AC signal between the first set of one or more first electrode elements 10L and the second set of one or more second electrode elements 10R and (b) application of the second AC signal between the third set of one or more third electrode elements 10A and the fourth set of one or more fourth electrode elements 10P varies over time. (For example, the orientation could vary over time between the situation depicted in the top left panel and top right panels of FIG. 5.)

In some embodiments of the apparatus depicted in FIGS. 4 and 5, each of the first electrode elements is capacitively coupled to the subject's body, each of the second electrode elements is capacitively coupled to the subject's body, each of the third electrode elements is capacitively coupled to the subject's body, and each of the fourth electrode elements is capacitively coupled to the subject's body. In alternative embodiments of the apparatus depicted in FIGS. 4 and 5, each of the first electrode elements is conductively coupled to the subject's body, each of the second electrode elements is conductively coupled to the subject's body, each of the third electrode elements is conductively coupled to the subject's body, and each of the fourth electrode elements is conductively coupled to the subject's body.

In some embodiments of the apparatus depicted in FIG. 4, the control signals generated by the controller 30 cause the AC signal generator 20 to vary the amplitude of both the first AC signal and the second AC signal over time so that that the orientation of the alternating electric field rotates. In alternative embodiments of the apparatus depicted in FIG. 4, the control signals generated by the controller 30 cause the AC signal generator 20 to vary the amplitude at least one of the first AC signal and the second AC signal over time so that the orientation of the alternating electric field oscillates back and forth. In these embodiments, because the overall directionality of the field can be controlled by adjusting the amplitude of the various components that contribute to the field, any desired speed of rotation (or oscillation) of the field can be implemented, including faster rotations/oscillations (e.g., 10 per second), slower rotations/oscillations (e.g., 10 per hour), or anywhere between those two rates. This stands in stark contrast to the situation described in U.S. Pat. No. 7,565,206, which is incorporated herein by reference, in which a rotating field is obtained by applying a sine wave to one pair of transducer arrays and applying a cosine wave to the other pair of transducer arrays, in which case the speed of rotation would be predetermined (and extremely fast).

In some embodiments of the apparatus depicted in FIG. 4, the control signals generated by the controller 30 cause the AC signal generator 20 to repeat the following steps in an alternating sequence at least 1000 times: setting the amplitudes of the first AC signal and the second AC signal so that the amplitude of the first AC signal is greater than the amplitude of the second AC signal; and setting the amplitudes of the first AC signal and the second AC signal so the amplitude of the second AC signal is greater than the amplitude of the first AC signal.

In some embodiments of the apparatus depicted in FIG. 4, the control signals generated by the controller 30 cause the AC signal generator 20 to repeat the following steps in an alternating sequence at least 1000 times: increasing the amplitude of the first AC signal from a minimum value to a maximum value; and decreasing the amplitude of the first AC signal from a maximum value to a minimum value.

Note that in the embodiments described above, the AC signal generator applies signal simultaneously to two pairs of transducer arrays, which changes the overall directionality between the four orientations 41-44 depicted in FIG. 5, and that all four of these overall directions 41-44 are positioned on a single two-dimensional plane. When additional pairs of transducer arrays are provided, the concepts described above can be extended to shift the overall directionality out of that single two-dimensional plane, and into any three dimensional overall directionality that is desired.

This can be accomplished, for example, by configuring the AC signal generator 20 to apply a third AC signal between a fifth set of one or more fifth electrode elements positioned on a fifth side of the target region and a sixth set of one or more sixth electrode elements positioned on a sixth side of the target region that is opposite to the fifth side, wherein the first, second, and third AC signals are all in phase with each other. The AC signal generator 20 is further configured to adjust an amplitude of the third AC signal based on additional control signals that arrive at the at least one control input.

In these three-dimensional embodiments, the controller 30 is further configured to send the additional control signals to the at least one control input, so that the control signals and the additional control signals collectively cause the AC signal generator 20 to vary the amplitude of the first, second, and third AC signals over time so that an orientation of an alternating electric field that is induced in the target region due to application of the first, second, and third AC signals varies over time in more than two dimensions.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method of applying an alternating electric field to a target region in a subject's body using a first set of one or more first electrode elements positioned on a first side of the target region, a second set of one or more second electrode elements positioned on a second side of the target region that is opposite to the first side, a third set of one or more third electrode elements positioned on a third side of the target region, and a fourth set of one or more fourth electrode elements positioned on a fourth side of the target region that is opposite to the third side, the method comprising:
inducing a first component of an electric field by applying a first AC signal between the first set of one or more first electrode elements and the second set of one or more second electrode elements;
inducing a second component of an electric field by applying a second AC signal between the third set of one or more third electrode elements and the fourth set of one or more fourth electrode elements, wherein the first and second AC signals are applied simultaneously, and wherein the first and second AC signals are in phase with each other; and
varying an amplitude of at least one of the first AC signal and the second AC signal over time such that an orientation of an alternating electric field formed by superposition of the first component and the second component varies over time.

2. The method of claim 1, further comprising:
positioning the first set of one or more first electrode elements on the first side of the target region;
positioning the second set of one or more second electrode elements on the second side of the target region;
positioning the third set of one or more third electrode elements on the third side of the target region; and
positioning the fourth set of one or more fourth electrode elements on the fourth side of the target region.

3. The method of claim 2, wherein each of the first electrode elements is capacitively coupled to the subject's body,
wherein each of the second electrode elements is capacitively coupled to the subject's body,
wherein each of the third electrode elements is capacitively coupled to the subject's body, and
wherein each of the fourth electrode elements is capacitively coupled to the subject's body.

4. The method of claim 2, wherein each of the first electrode elements is conductively coupled to the subject's body,
wherein each of the second electrode elements is conductively coupled to the subject's body,
wherein each of the third electrode elements is conductively coupled to the subject's body, and
wherein each of the fourth electrode elements is conductively coupled to the subject's body.

5. The method of claim 1, wherein the varying comprises varying the amplitudes of both the first AC signal and the second AC signal over time so that that the orientation of the alternating electric field rotates.

6. The method of claim 1, wherein the varying comprises varying the amplitude of at least one of the first AC signal and the second AC signal over time so that the orientation of the alternating electric field oscillates back and forth.

7. The method of claim 1, wherein the varying comprises repeating the following steps in an alternating sequence at least 1000 times:
setting the amplitudes of the first AC signal and the second AC signal so that the amplitude of the first AC signal is greater than the amplitude of the second AC signal; and
setting the amplitudes of the first AC signal and the second AC signal so the amplitude of the second AC signal is greater than the amplitude of the first AC signal.

8. The method of claim 1, wherein the varying comprises repeating the following steps in an alternating sequence at least 1000 times:
increasing the amplitude of the first AC signal from a minimum value to a maximum value; and
decreasing the amplitude of the first AC signal from a maximum value to a minimum value.

9. The method of claim 1, further comprising inducing a third component of an electric field by applying a third AC signal between a fifth set of one or more fifth electrode elements positioned on a fifth side of the target region and a sixth set of one or more sixth electrode elements positioned on a sixth side of the target region that is opposite to the fifth side,
wherein the first, second, and third AC signals are all applied simultaneously, and wherein the first, second, and third AC signals are all in phase with each other; and
wherein an amplitude of the first, second, and third AC signals are varied over time such that an orientation of a vector representing a superposition of the first component, the second component, and the third component varies over time in more than two dimensions.

10. An apparatus for applying an alternating electric field to a target region in a subject's body using a first set of one or more first electrode elements positioned on a first side of the target region, a second set of one or more second electrode elements positioned on a second side of the target region that is opposite to the first side, a third set of one or more third electrode elements positioned on a third side of the target region, and a fourth set of one or more fourth electrode elements positioned on a fourth side of the target region that is opposite to the third side, the apparatus comprising:
an AC signal generator configured to apply a first AC signal between the first set of one or more first electrode elements and the second set of one or more second electrode elements and to simultaneously apply a second AC signal between the third set of one or more third electrode elements and the fourth set of one or more fourth electrode elements, wherein the first and second AC signals are in phase with each other, and wherein the AC signal generator is configured to adjust an amplitude of the first AC signal and to adjust an amplitude of the second AC signal based on control signals that arrive at at least one control input; and
a controller configured to send a sequence of control signals to the at least one control input, wherein the control signals cause the AC signal generator to vary the amplitude of at least one of the first AC signal and the second AC signal over time so that an orientation of an alternating electric field that is induced in the target region due to (a) application of the first AC signal between the first set of one or more first electrode elements and the second set of one or more second electrode elements and (b) application of the second AC signal between the third set of one or more third electrode elements and the fourth set of one or more fourth electrode elements varies over time.

11. The apparatus of claim 10, further comprising:
the first set of one or more first electrode elements;
the second set of one or more second electrode elements;
the third set of one or more third electrode elements; and
the fourth set of one or more fourth electrode elements.

12. The apparatus of claim 11, wherein each of the first electrode elements is capacitively coupled to the subject's body,
wherein each of the second electrode elements is capacitively coupled to the subject's body,
wherein each of the third electrode elements is capacitively coupled to the subject's body, and
wherein each of the fourth electrode elements is capacitively coupled to the subject's body.

13. The apparatus of claim 11, wherein each of the first electrode elements is conductively coupled to the subject's body,
wherein each of the second electrode elements is conductively coupled to the subject's body,
wherein each of the third electrode elements is conductively coupled to the subject's body, and
wherein each of the fourth electrode elements is conductively coupled to the subject's body.

14. The apparatus of claim 10, wherein the control signals cause the AC signal generator to vary the amplitude of both the first AC signal and the second AC signal over time so that that the orientation of the alternating electric field rotates.

15. The apparatus of claim 10, wherein the control signals cause the AC signal generator to vary the amplitude at least one of the first AC signal and the second AC signal over time so that the orientation of the alternating electric field oscillates back and forth.

16. The apparatus of claim 10, wherein the control signals cause the AC signal generator to repeat the following steps in an alternating sequence at least 1000 times:
setting the amplitudes of the first AC signal and the second AC signal so that the amplitude of the first AC signal is greater than the amplitude of the second AC signal; and
setting the amplitudes of the first AC signal and the second AC signal so the amplitude of the second AC signal is greater than the amplitude of the first AC signal.

17. The apparatus of claim 10, wherein the control signals cause the AC signal generator to repeat the following steps in an alternating sequence at least 1000 times:
increasing the amplitude of the first AC signal from a minimum value to a maximum value; and
decreasing the amplitude of the first AC signal from a maximum value to a minimum value.

18. The apparatus of claim 10, wherein the AC signal generator is further configured to apply a third AC signal between a fifth set of one or more fifth electrode elements positioned on a fifth side of the target region and a sixth set of one or more sixth electrode elements positioned on a sixth side of the target region that is opposite to the fifth side, wherein the first, second, and third AC signals are all in phase with each other, and wherein the AC signal generator is further configured to adjust an amplitude of the third AC signal based on additional control signals that arrive at the at least one control input; and
wherein the controller is further configured to send the additional control signals to the at least one control input, wherein the control signals and the additional control signals collectively cause the AC signal generator to vary the amplitude of the first, second, and third AC signals over time so that an orientation of an alternating electric field that is induced in the target region due to application of the first, second, and third AC signals varies over time in more than two dimensions.
